(19) United States
(12) Patent Application Publication
(10) Pub. No.: US 2025/0312382 A1
(43) Pub. Date: Oct. 9, 2025

(54) Cell Therapy for Treating Liver Disease

(71) Applicant: HepaTx Corporation, Palo Alto, CA (US)

(72) Inventor: Eric Schuur, Bend, OR (US)

(73) Assignee: HepaTx Corporation, Palo Alto, CA (US)

(21) Appl. No.: 19/092,716

(22) Filed: Mar. 27, 2025

Related U.S. Application Data
(60) Provisional application No. 63/574,884, filed on Apr. 4, 2024.

(51) Int. Cl. A61K 35/407; A61K 47/02; A61K 47/42; A61P 1/16

(52) U.S. Cl. CPC A61K 35/407; A61K 47/02; A61K 47/42; A61P 1/16

(57) ABSTRACT

The present disclosure provides Hx.001, and methods of using Hx.001 to treat liver disease. In an aspect, Hx.001 can regenerate liver tissue.

### BACKGROUND

Morbidity and mortality from liver disease are very high. Mortality from liver disease ranks 12th in the United States. Nearly 4% of hospitalizations in the US are for chronic liver disease at a cost of nearly $20 billion.2 Alcohol-related hepatitis (AH) and non-alcoholic steatohepatitis (NASH), contribute more than 50% to the burden of chronic liver disease in the United States. Orthotopic liver transplantation is an effective treatment but is severely limited in availability.

It is estimated that fully one-third of the population of the United States of America has some type of liver disease. 1 Nearly 4% of hospitalizations in the US are for chronic liver disease at a cost of nearly $20 billion.2 Alcohol-related hepatitis, metabolic dysfunction associated steatohepatitis, viral hepatitis, and autoimmune hepatitis all contribute to the burden of chronic liver disease.

Hepatocyte loss, inflammation, and fibrosis are characteristic of the pathology of chronic liver disease regardless of the etiology. Infusion of suspensions of mesenchymal stromal cells (MSCs) can address multiple aspects of this pathology.3,4 Our data shows that this type of cell therapy can 1) reduce inflammatory macrophage activity, 2) promote reparative macrophage activity, 3) suppress CD4+ and CD8+ T cell activation, and 4) stimulate Treg differentiation.4-9 In addition, MSC therapy has been demonstrated to promote the resolution of liver fibrosis in both animal and human studies by influencing hepatic stellate cell activity, increased vascularization, and extracellular matrix remodeling.10-16 Conversion to hepatocyte-like cells (HLCs) prior to treatment amplifies these properties by increasing secretion of hepatotropic factors and promoting homing to the liver.17-19

### SUMMARY

Mesenchymal stem/stromal cells (MSCs) have emerged as a promising therapeutic modality in multiple chronic diseases, including liver disease. However, despite numerous clinical evaluations, only mixed success has been reported when used as a cell therapy in several types of clinical applications. There seem to be two main sources of this variability. The first is donor to donor variability in MSC function (intrinsic variability). The second is a lack of clear understanding of process variables for MSCs production that affect potency (i.e. critical quality attributes (CQAs)) and consequently variable potency of the clinical product (extrinsic variability). The hypothesis underlying this work is that both sources of variability are reflected by variations in gene expression. The proposed work will identify the drivers of variability by building detailed transcriptomic databases of MSCs and derived products. In this proposal the test system will be adipose tissue derived MSCs, hepatocyte-like cells derived from those MSCs, and a liver-relevant functional outcome measure. A successful outcome will significantly advance development of MSC-derived cell therapies for liver disease and potentially other chronic diseases.

MSC-based cell therapies offer the opportunity to treat both forms of liver disease. Ample evidence from published studies demonstrates the potential for cell therapy to have a positive clinical benefit in NASH and AH. This approach offers several advantages relative to liver transplant, including much greater availability, lower morbidity, and dramatically lower cost. However, for MSC-based cell therapy to deliver on this promise the problem of consistent potency must be addressed. Inconsistent potency both impedes development by necessitating greater numbers of clinical studies and drives up the ultimate cost of the therapies by increasing development costs and increasing manufacturing costs due to production failures. The field has struggled to address extrinsic variability and its effects on potency. Studies have shown that treatment of MSCs with inflammatory cytokines (“licensing” with interferon gamma and tumor necrosis factor alpha) can increase the potency of MSCs with respect to their immunomodulatory properties but this does not affect variability in function. So far, no methods for addressing intrinsic variability have been advanced.

Hepatx is developing manufacturing methods that address the potency problem directly and increase their potency in liver disease. Preliminary data suggest that the current methods can reduce both extrinsic (production-related) variability and intrinsic (donor-to-donor) variability in the final product. The proposed work will build a foundation to understand how to further improve potency and reduce variability between batches and between donors. To do this we will build transcriptomic databases using single cell RNA sequencing coupled to function output to develop a detailed understanding of the genes driving functional variability between MSC donors and between HLC batches. Detailed understanding of genes driving variability will provide information on the mechanism of action of HLCs in liver disease, enabling further improvements in potency. These same data will inform CQA selection and enable development of more efficient production methods.

### BRIEF DESCRIPTION OF THE FIGURES

FIG. 1A shows HX001 spheres made from 2,000 Hx-001 cells. FIG. 1B shows the developmental trajectory UMAP.

FIG. 2 shows the reduction of inflammatory cytokine production from co-culture of Hx-001 cells with activated macrophages.

FIG. 3A, FIG. 43B and FIG. 3C show results from an animal model for liver injury. FIG. 3A shows effect on ALT levels in the blood of mice treated with Hx-001. FIG. 3B shows effect on inflammatory cytokines in the liver of mice treated with Hx-001. FIG. 3C shows the effect on smooth muscle actin and collagen I in liver tissue of mice treated with Hx-001.

FIG. 4 shows the release of oxygen from islets in a cryogel-CPO bioscaffold.

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

As used in this specification and the appended claims, the singular forms “a”, “an” and “the” include plural referents unless the context clearly indicates otherwise. Thus, for example, reference to “a polypeptide” includes more than one polypeptide.

The section headings used herein are for organizational purposes only and not to be construed as limiting the subject matter described.

### Definitions

As used herein, the terms “protein”, “polypeptide,” and “peptide” are used interchangeably and are defined to mean a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids. In some embodiments of the descriptions of polypeptides, the standard single or three letter abbreviations are used for the genetically encoded amino acids (see, e.g., IUPAC-IUB Joint Commission on Biochemical Nomenclature, “Nomenclature and Symbolism for Amino Acids and Peptides,” Eur. J. Biochem. 138:9-37, 1984).

As used herein, the terms “polynucleotide” or “nucleic acid’ are used interchangeably and are defined to mean two or more nucleosides that are covalently linked together. The polynucleotide may be wholly comprised ribonucleosides (i.e., an RNA), wholly comprised of 2′ deoxyribonucleotides (i.e., a DNA) or mixtures of ribo- and 2′ deoxyribonucleosides. While the nucleosides will typically be linked together via standard phosphodiester linkages, the polynucleotides may include one or more non-standard linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. Preferably, such modified or synthetic nucleobases will be encoding nucleobases.

As used herein, the term “coding sequence” is defined to mean a portion of a nucleic acid (e.g., a gene) that encodes an amino acid sequence of a protein.

As used herein, the terms “wild-type” is defined to mean the form found predominantly in nature. For example, a wild-type polypeptide or polynucleotide sequence is a sequence predominantly present in an organism that can be isolated from a source in nature and which has not been intentionally modified by human manipulation.

As used herein, the terms “recombinant” or “engineered” or “non-naturally occurring” are used interchangeably and are defined to mean modified polypeptides or nucleic acids which polypeptides or nucleic acids are modified in a manner that would not otherwise exist in nature, or is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

As used herein, the terms “percentage of sequence identity” and “percentage homology” are used interchangeably and are defined to mean comparisons among polynucleotides or polypeptides, and are determined by comparing two optimally aligned sequences over a comparison window, where the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, Adv Appl Math. 2:482, 1981; by the homology alignment algorithm of Needleman and Wunsch, J Mol Biol. 48:443, 1970; by the search for similarity method of Pearson and Lipman, Proc Natl Acad Sci. USA 85:2444, 1988; by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Greene Publishing Associates, Inc. and John Wiley & Sons, Inc., (1995 Supplement). Examples of algorithms that are suitable for determining percent sequence identity and sequence similarity are the BLAST and BLAST 2.0 algorithms, which are described in Altschul et al., J. Mol. Biol. 215:403-410, 1990; and Altschul et al., Nucleic Acids Res. 25 (17): 3389-3402, 1977; respectively. Software for performing BLAST analyses is publicly available through the National Center for Biotechnology Information website. BLAST for nucleotide sequences can use the BLASTN program with default parameters, e.g., a wordlength (W) of 11, an expectation (E) of 10, M=5, N=−4, and a comparison of both strands. BLAST for amino acid sequences can use the BLASTP program with default parameters, e.g., a wordlength (W) of 3, an expectation (E) of 10, and the BLOSUM62 scoring matrix (see Henikoff and Henikoff, Proc Natl Acad Sci. USA 89:10915, 1989). Exemplary determination of sequence alignment and % sequence identity can also employ the BESTFIT or GAP programs in the GCG Wisconsin Software package (Accelrys, Madison WI), using default parameters provided.

As used herein, the term “reference sequence” is defined to mean a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes to the primary sequence.

As used herein, the term “substantial identity” refers to a polynucleotide or polypeptide sequence that has at least 80 percent sequence identity, at least 85 percent identity and 89 to 95 percent sequence identity, more usually at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In specific embodiments applied to polypeptides, the term “substantial identity” means that two polypeptide sequences, when optimally aligned, such as by the programs GAP or BESTFIT using standard parameters, i.e., default parameters, share at least 80 percent sequence identity, preferably at least 89 percent sequence identity, at least 95 percent sequence identity or more (e.g., 99 percent sequence identity). Preferably, residue positions which are not identical differ by conservative amino acid substitutions.

As used herein, the terms “corresponding to”, “reference to” or “relative to” are used interchangeably when used in the context of the numbering of a given amino acid or polynucleotide sequence and are defined in this context to mean the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered ______, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned. As such, the term “corresponding to”, “reference to” or “relative to” also refers to a residue that is analogous, homologous, or equivalent to an enumerated residue in a reference polypeptide. In addition, in some embodiments, crystal structure coordinates of a reference sequence may be used as an aid in determining a homologous polypeptide residue's three dimensional structure and location of equivalent residues.

As used herein, the terms “consensus sequence” and “canonical sequence” are defined to mean an archetypical amino acid sequence against which all variants of a particular protein or sequence of interest are compared. The terms also refer to a sequence that sets forth the nucleotides that are most often present in a DNA sequence of interest. For each position of a gene, the consensus sequence gives the amino acid that is most abundant in that position in a multiple sequence alignment (MSA).

As used herein, the terms “optimal alignment” or “optimally aligned” are defined to mean the alignment of two (or more) sequences giving the highest percent identity score. For example, optimal alignment of two polypeptide sequences can be achieved by aligning the sequences such that the maximum number of identical amino acid residues in each sequence are aligned together or by using software programs or procedures described herein or known in the art. Optimal alignment of two nucleic acid sequences can be achieved by aligning the sequences such that the maximum number of identical nucleotide residues in each sequence are aligned together. Two sequences (e.g., polypeptide sequences) may be deemed “optimally aligned” when they are aligned using defined parameters, such as a defined amino acid substitution matrix, gap existence penalty (also termed gap open penalty), and gap extension penalty, so as to achieve the highest similarity score possible for that pair of sequences. Optimal alignment can be done manually or by using software programs or procedures described herein or known in the art. e.g., the BLASTP program for amino acid sequences and the BLASTN program for nucleic acid sequences.

As used herein, the terms “amino acid substitution” or “amino acid difference” are defined to mean a change in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based.

As used herein, the terms “conservative amino acid substitution” or “conservative amino acid difference” are defined to mean a change in the amino acid at a residue position to a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid, e.g., alanine, valine, leucine, and isoleucine; an amino acid with hydroxyl side chain is substituted with another amino acid with a hydroxyl side chain, e.g., serine and threonine; an amino acid having aromatic side chains is substituted with another amino acid having an aromatic side chain, e.g., phenylalanine, tyrosine, tryptophan, and histidine; an amino acid with a basic side chain is substituted with another amino acid with a basic side chain, e.g., lysine and arginine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively. Exemplary conservative substitutions are provided in Table 1 below.

TABLE 1

| Residue | Possible Conservative Substitutions |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| N, Q, S, T | Other polar |
| H, Y, W, F | Other aromatic (H, Y, W, F) |
| C, P | None |

As used herein, the terms “non-conservative substitution” or “non-conservative amino acid difference” are defined to mean a change in the amino acid at a residue position to a different residue with significantly differing side chain properties. Non-conservative substitutions may use amino acids between, rather than within, the defined groups and affects (a) the structure of the peptide backbone in the area of the substitution (e.g., proline for glycine), (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As used herein, the term “deletion” is defined to mean a modification of a polypeptide by removal of one or more amino acids from the reference polypeptide or modification of a nucleic acid by removal of one or more nucleotides from the reference nucleic acid.

As used herein, the term “insertion” is defined to mean a modification to a polypeptide by addition of one or more amino acids from the reference polypeptide, or modification of a nucleic acid by addition of one or more nucleic acids.

As used herein, the term “gene” is defined to mean a polynucleotide (e.g., a DNA segment) that encodes a polypeptide. The term includes regions preceding and following the coding regions as well as any intervening sequences when present (e.g., introns) between individual coding segments (exons).

As used herein, the term “homologous genes” is defined to mean a pair of genes which correspond to each other and which are identical or similar to each other. The term encompasses genes that are separated by speciation (i.e., the development of new species) (e.g., orthologous genes), as well as genes that have been separated by genetic duplication (e.g., paralogous genes).

As used herein, the terms “ortholog” and “orthologous genes” are defined to mean genes in different species that have evolved from a common ancestral gene (i.e., a homologous gene) by speciation. Typically, orthologs retain the same function during the course of evolution. Identification of orthologs finds use in the reliable prediction of gene function in newly sequenced genomes.

As used herein, the terms “paralog” and “paralogous genes” are defined to mean genes that are related by duplication within a genome. Generally, paralogs tend to evolve into new functions, even though some functions are often related to the original one.

As used herein, the term “chromosomal integration” is defined to mean the process whereby an incoming sequence is introduced into the chromosome of a host cell. The homologous regions of the transforming DNA align with homologous regions of the chromosome. Subsequently, the sequence between the homology boxes is replaced by the incoming sequence in a double crossover (i.e., homologous recombination). In some embodiments, homologous sections of an inactivating chromosomal segment of a DNA construct align with the flanking homologous regions of the indigenous chromosomal region of a host cell chromosome. Subsequently, the indigenous chromosomal region is deleted by the DNA construct in a double crossover (i.e., homologous recombination).

As used herein, the term “homologous recombination” is defined to mean the exchange of DNA fragments between two DNA molecules or paired chromosomes at the site of identical or nearly identical nucleotide sequences. In some embodiments, chromosomal integration is homologous recombination.

As used herein, the term “stringent hybridization conditions” is defined to mean hybridizing in 50% formamide at 5×SSC at a temperature of 42° C. and washing the filters in 0.2×SSC at 60° C. (1×SSC is 0.15M NaCl, 0.015M sodium citrate.) Stringent hybridization conditions also encompasses low ionic strength and high temperature for washing, for example 0.015 M sodium chloride/0.0015 M sodium citrate/0.1% sodium dodecyl sulfate at 50° C.; hybridization with a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

As defined herein, the term “heterologous” polynucleotide or polypeptide is defined to mean any polynucleotide or polypeptide that is not naturally found in a host cell. As such, the term includes polynucleotides that are removed from a host cell, subjected to laboratory manipulation, and then reintroduced into a host cell. In some embodiments, the introduced polynucleotide expresses the heterologous polypeptide.

As used herein, the term “codon optimized” is defined to mean changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome.

As used herein, the term “control sequence” is defined to include all components, which are necessary or advantageous for the expression of a polynucleotide and/or polypeptide of the present disclosure. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and where appropriate, translational stop signals. The control sequences may be provided with linkers for the purpose of introducing specific restriction sites facilitating ligation of the control sequences with the coding region of the nucleic acid sequence encoding a polypeptide.

As used herein, the term “operably linked” is defined to mean a configuration in which a control sequence is appropriately placed (i.e., in a functional relationship) at a position relative to a polynucleotide of interest such that the control sequence directs or regulates the expression of the polynucleotide and/or polypeptide of interest.

As used herein, the term “promoter sequence” is defined to mean a nucleic acid sequence that is recognized by a host cell for expression of a polynucleotide of interest, such as a coding sequence or gene. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

### Mesenchymal Stem Cells and Liver Treatment

MSC therapies delivered as cell suspensions are cleared by phagocytosis within several days after administration.20-22 This may limit the potency of the therapy and necessitate multiple infusions. Lack of trophic factors that promote cell survival, lack of access to appropriate cellular niches, and acute hypoxia contribute to cell loss.23-25 Strategies to overcome cell loss that have been tried including the formation of cellular aggregates (spheroids) and the combination of cells or spheroids with biocompatible materials to improve survival in vivo.24,26 Both of these strategies seek to provide trophic factors for survival and/or shielding from immune clearance.

We will combine HLCs (Hx-001 from Hepatx) with bioscaffolds that provide an environment conducive to cell survival, engraftment, and extended function. This novel combination has never been tried before. Hx-001 cells have repeatedly demonstrated promise in treating multiple types of liver disease (reviewed in Bogliotti et al. 2022 3). These cells represent a novel treatment approach to liver disease that acts by multiple mechanisms, including secreting anti-inflammatory and pro-regenerative factors. We have shown that administration of a suspension of Hx-001 cells has a positive impact on clinically relevant parameters in multiple liver disease models in mice, including carbon tetrachloride treatment, alcohol feeding, and acetaminophen overdose (see below). Hx-001 is a cell suspension that is scalable and cost-effective to produce.

A suspension of Hx-001 cells in buffer are almost entirely cleared in mice within 48 hours, which is consistent with published results.20 Similarly, in humans, cell suspensions given IV are cleared within 1-2 days.21,22 By extending the viability of cell therapies in vivo can increase their potency. Given our goal of extended viability in vivo, collagen, which has been shown to facilitate the survival of MSCs and works well with Hx-001, is an excellent choice that has been shown to facilitate in vivo survival.27 Hepatocyte viability in vitro for up to 7 days has been demonstrated with fluorocarbon-based oxygen carrier in gelled collagen.28 The Thakor Group has developed novel collagen-based bioscaffolds that can improve long-term survival of pancreatic islet cell therapy in vivo for up to 30 days and lead to an improved therapeutic effect.29 These bioscaffolds may address the need for structural and trophic support for Hx-001 spheroids by providing an attachment matrix and providing long-term trophic support through improved oxygen generation during vascularization.

Our hypothesis is that providing trophic and structural support will improve the survival of the Hx-001 spheroids in vivo, allowing them to secrete therapeutic factors over a longer time, and thereby improve their therapeutic effect. Combination with a bioscaffold will also enable localized treatment, further increasing therapeutic effect.

Biomaterials for improving cell therapies have been tested with other cell therapies, including pancreatic islet transplantation to treat diabetes, hepatocytes to treat acute liver failure, and MSCs to treat inflammatory diseases.26,31-33 For example, current practice for select diabetic patients is the intrahepatic delivery of isolated islets. While this treatment can restore normoglycemia, islets are often rapidly lost to mechanical stress and immune rejection. Several studies have been published that use bioscaffolds to provide physical support, protection and, in some cases, trophic molecules to enhance the effectiveness/potency of transplanted islets. A variety of materials have been tested including collagen, and poly(dimethylsiloxane) (PDMS).29,34,35 The reported results demonstrated improved glycemic control and islet survival. Biomaterials have also been used to deliver mature hepatocytes as a treatment for liver disease.26,31, to increase half-life of MSCs following intravenous injection 33, and improve the survival and function of MSC spheres in vivo.16

Over the last several years, the Thakor Group has developed a collagen-based bioscaffold strategy for enhancing cell survival in vivo and applied this technology to islet transplantation.29,36,37 What makes this approach unique is that the technology enables titration of calcium peroxide (CPO) into the matrix during the cryogelation step ensuring that all cells seeded into the cryogel-CPO bioscaffold will have, for the first time, a uniform supply of oxygen. This has advantages over other technologies which (i) cannot ensure an even and uniform delivery of oxygen (i.e. the PDMS-CaO2 (Oxysite), (ii) use platforms that induce inflammatory reactions (i.e. perfluorodecalin (PFD)-plasma constructs), (iii) involve the addition of microorganisms (i.e. Chlorella sorokiniana) or need oxygen reservoirs to be continually replenished (i.e. BAir technologies). Hepatx is prepared to license intellectual property covering this technology for application with Hx-001.

Bioscaffolds will be synthesized with 0 wt %, 0.25 wt %, 0.5 wt %, and 1.0 wt % CPO, the range of CPO concentrations used in previous studies.29 Spheres averaging approximately 2000 Hx-001 cells formed in suspension will be used. Cell loading will be performed in microwell plates using 20-200 spheres of Hx-001 cells per bioscaffold. Cellular viability will be assessed at 24 hours. Spheres that have been incubated in the absence of bioscaffolds will be used as a control for viability.

Bioscaffold synthesis: bioscaffolds will be synthesized from bovine achilles tendon collagen (Sigma-Aldrich) as described.29 Briefly, collagen is dispersed in 5 mM hydrochloric acid (HCl) overnight at 4° C., then mixed with CPO (Sigma Aldrich) to achieve the desired wt %, placed in molds, and crosslinking initiated by addition of N-hydroxysuccinimide and 1-ethyl-3-3 (3-dimethylaminopropyl) carbodiimide hydrochloride. The mixtures in the molds are placed at −20° C. until solid. Subsequently, the collagen gel-CPO mixtures are thawed and washed with distilled H2O and cut to size. These bioscaffolds with 300 um pores will be used for the studies described. Bioscaffold porosity, density, structure, and mechanical properties will be analyzed as described.29 We will validate oxygen and oxygen free radical generation as described.29

SF-Heps will be differentiated in vitro from ASCs using our proprietary culture methods. Briefly, ASCs will be isolated from human lipoaspirate, then placed in suspension culture in a 125 mL Corning spinner flask in Dulbecco's modified Eagle medium+10% fetal bovine serum (FBS) and stirred at 40 rpm. Following overnight culture, sphere formation will be confirmed and the spheres transferred to stage 1 medium for differentiation into definitive endoderm. After two days of culture, the spheres are transferred to stage 2 medium (William's E with hepatocyte growth factor (HGF), fibroblast growth factor 4 (FGF4), epidermal growth factor (EGF), dexamethasone, and dimethyl sulfoxide) and cultured for an additional 6 days. Hepatic differentiation will be confirmed by quantitative PCR for selected hepatocyte specific genes and function will be evaluated by ability to suppress inflammatory cytokine expression in co-cultures with human activated macrophages as described. 17 Two batches of Hx-001 cells will be produced that meet Hepatx established QC criteria: expression of benchmark genes (AAT, AGT, KRT18, SOD2, HGF, GLUL) within 2-fold of average from our database of >30 differentiations and >50% reduction in expression of IL-1 beta and TNF alpha in macrophage co-cultures.

Hx-001 spheres will be seeded into sterilized bioscaffolds, achieving a density of 20-200 spheres in 200 μL complete medium per bioscaffold; these will be placed in each well of a 96-well plate. Spheres seeded into bioscaffolds will be cultured in a humidified incubator with 20% 02 and 5% CO2 at 37° C. for 7 days prior to analysis. Quadruplicate samples of each bioscaffold-Hx-001 pairing and controls will be produced and analyzed.

The phenotype of the Hx-001 cells in the bioscaffolds will be compared to those without bioscaffolds by RNA sequencing followed by principle component (PC) analysis using JMP 14.0. Averages of the PC1 and PC2 dimentions will be compared across samples using Student's T test with p<0.05 considered statistically significant. The viability and function of Hx-001 in the scaffolds will be determined using 3-(4,5-dimethylthiazol2-yl)-2,5-diphenyltetrazolium bromide (MTT) assay as directed by the manufacturer (Abcam). Unloaded bioscaffolds will be used as negative controls and equal numbers of Hx-001 spheres cultured in the absence of bioscaffolds will be used as positive controls. Live/dead imaging will be performed by incubating bioscaffold-sphere combination with fluorescein diacetate and propidium iodide (Thermo Fisher Live-Dead stain) for 20 minutes followed by confocal imaging. Triplicate samples of cell-loaded bioscaffolds will be tested and the experiment will be run twice to confirm results.

We are replicating published conditions for the cryogel bioscaffold formation, so we anticipate similar characteristics for physical properties and oxygen/oxygen radical generation using the methods here. We expect gene expression PC1 and PC2 for Hx-001 in bioscaffolds to be within one standard deviation of cells without bioscaffolds. Under the conditions described sphere formation is predictable and yields a normal distribution of sphere of Hx-001 cells. If we determine that additional control over sphere size is desired, we have also successfully generated uniform size spheres using Aggrewells (StemCell Technologies). We anticipate that Hx-001 spheres will readily attach to bioscaffolds since the spheres readily attach to collagen coated surfaces and that viability will be comparable to spheres in the absence of cryogels. The CPO wt %-sphere number combination that maximizes number of viable cells as measured by MTT assay will be used in subsequent experiments. If similar maximum viable sphere number is seen with multiple combinations, the combination with the lowest CPO wt % will be used. The target viability will be 90% of controls. Triplicate samples at each concentration will be measured. Means for MTT assay will be compared by Student's T test with p=0.05 used as the level of significance. If the collagen-based bioscaffolds do not support attachment of viable Hx-001 cells we will repeat the loading experiments with graphene scaffolds that have been shown to accommodate MSCs effectively.36

We will evaluate retention of functional properties for the Hx-001 cells in the sphere-loaded bioscaffolds. Hx-001 cells manufactured from ASCs will be loaded into CPO bioscaffolds using optimal conditions from Aim 1, and then tested for cytokine secretion and immunomodulatory properties in macrophage co-cultures.

Triplicate Hx-001 loaded bioscaffolds will be incubated in complete Hx-001 stage 2 medium without hepatocyte growth factor in microwell plates for 48 hours. Medium will then be collected, centrifuged to remove debris, and snap frozen in liquid nitrogen and stored at −80° C. Aliquots will be submitted for cytokine measurement at the Stanford Human Immune Monitoring Core. Cytokine concentrations will be measured using Luminex technology using the Luminex Human 80-plex assay (custom from EMD-Millipore). Equivalent numbers of Hx-001 spheres without bioscaffolds will be incubated in parallel as controls in triplicate. The experiment will be repeated to confirm results.

Hx-001-loaded or empty bioscaffolds, as well as Hx-001 spheres without bioscaffolds will co-cultured with U937 monocyte cultures to evaluate the effects of the scaffolds on synthesis of cytokines by macrophages. Briefly, U937 cells will be differentiated to M0 macrophages using 4α-phorbol 12-myristate 13-acetate (PMA) overnight in Iscove's Modified Dulbecco Medium plus 10% fetal bovine serum (complete macrophage medium) at 37° C. To induce the inflammatory (M1) macrophage phenotype, the M0 macrophage cultures will be incubated with complete macrophage medium containing 100 ng/mL lipopolysaccharide and 20 ng/mL interferon gamma. After a 3 hour incubation the Hx-001 loaded bioscaffolds will be added to the macrophage cultures using Transwells. Incubation will continue for an additional 21 hours. Co-cultures will be terminated by lysis of the macrophages in RNA lysis buffer. RNA purified from the co-cultures will be analyzed for human cytokine expression by qPCR. The experiment will be repeated once to confirm the results.

We anticipate that loading of Hx-001 spheres in scaffolds containing CPO will not adversely affect the functional properties of the cells as measured by cytokine secretion profile and co-cultures with macrophages. We expect to observe secretion of HGF, VEGFA, and CCL2 at similar levels as Hx-001 spheres free of bioscaffold material. Success will be considered expression within 1 standard deviation of scaffold-free spheres in replicate experiments. In macrophage co-cultures, we routinely observe reductions in IL-1 beta by 76%, and TNF alpha by 82% after 21 hours of co-culture, which we expect to replicate with Hx-001 cells loaded on bioscaffolds. Co-culture experiments have a greater level of variability than gene expression assay. We will consider successful results to be a reduction of 50% in both IL-1 beta and TNF alpha from the M1 macrophages for spheres with and without bioscaffolds. If we do not observe similar results with the combination, it may be that the bioscaffold causes dedifferentiation. Potential solutions include differentiation of the Hx-001 cells under conditions with 5-aza cytidine known to enhance differentiation (Hepatx unpublished), derivatizing the scaffold material with trophic factors, or testing graphene bioscaffolds as described in Aim 1.38.

Our hypothesis is that Hx-001 cells embedded in bioscaffolds will secrete therapeutic molecules and impact pathophysiological processes in liver disease. To evaluate this hypothesis bioscaffolds with or without Hx-001 cells will be implanted in the inguinal fat pad of NSG mice chronically treated with carbon tetrachloride. At 48 hours and 7 days post-implantation blood samples will be analyzed for human cytokines and explanted bioscaffolds will be analyzed for functional Hx-001 cells and host response.

NOD SCID mice (50% male and 50% female, Charles River) 6-7 weeks old will be maintained under aseptic conditions in barrier cages. Groups of 12 mice will be tested in four groups: (1) background control (mock surgery), (2) omentum ‘implanted with Hx-001 spheres without bioscaffold, (3) omentum implanted bioscaffold with cells for 7 day harvest, (4) omentum implanted bioscaffold with cells for 21 day harvest. Liver injury will be induced by treatment with CC14 as this model more reliably induces inflammation and fibrosis than other models we use. Hepatx has extensive experience with this model. Mice will receive biweekly doses of 0.5 μL/gm of CC14 in mineral oil subcutaneously for 8 weeks. At 8 weeks surgery will be performed to implant bioscaffolds. Hx-001 loaded bioscaffolds (with 125 spheres (250,000 cells) or the optimal loading from Aim 1 per side will be used. The mice will be anesthetized with 2% isoflurane in oxygen. The abdomen will be shaved and swabbed with betadine and isopropanol to sterilize the skin. An incision will be made through the peritoneal wall in the midline. The omentum will be used as it is accessible and vascularized. The omentum will be gently pulled out from the abdominal cavity using forceps. A bioscaffold will be placed on the unfolded omentum. The omentum will then folded and sutured around the bioscaffold before being placed back into the abdominal cavity of the animal. The wound will be closed with surgical clips. Mice will be allowed to recover for 24 hours before continuing CC14 injections. Group (3) mice will be sacrificed at 7 days after surgery. The remaining mice will be sacrificed at 21 days post-surgery.

At sacrifice, blood will be collected, allowed to clot, and serum stored at −80° C. Scaffolds and their associated omentum, livers, lungs, and kidneys will be harvested; liver left lobe, one lung, and one kidney will be snap frozen for nucleic acid and histologic analysis. The remaining tissue will be fixed using paraformaldehyde for histologic analysis. Sections will be prepared for histological and immunohistochemical analyses to determine Hx-001 sphere structure, viability, and vascularization (Haemotoxylin and Eosin (H&E), Mason's trichrome, Ki67, and CK18 staining). The stained sections will then be imaged using a NanoZoomer slide scanner 2.0-RS (Hamamatsu, Japan). Results will be analyzed using FIJI Image J software for 5 different sections per animal. Serum samples will be analyzed for HGF, CCL2, and VEGFA by ELISA specific for human proteins (Abcam #ab275901, #ab179886, #ab 119566). Serum ALT and AST will also be analyzed to evaluate for changes in therapeutic efficacy of the transplanted cells.

We expect to be able to detect one or more of the following: HGF, VEGFA, and CCL2 by ELISA in serum from mice with Hx-001-bioscaffolds implanted. The average of triplicates for expression of these cytokines will be compared using Student's T test with p=0.05 used as a significance level. If no cytokines are detected, we may subject samples to Luminex analysis as in Aim 2 to survey for other human cytokines in the case where the cytokines we tested for were specifically not expressed. We will also immunostain sections of the bioscaffolds for the cytokines above to determine if they are being synthesized but are not entering the bloodstream. H&E sections of the bioscaffold-cell combinations will reveal the integrity of the Hx-001 cells in the bioscaffolds, as well as demonstrating any murine cell infiltration and/or foreign body reaction. Sections will be reviewed with a board-certified pathologist to evaluate for cell integrity, infiltrates, and vascularization. Similarly, liver sections will be scored by a pathologist for inflammatory infiltrate and fibrosis. Pearson's Chi2 test will be used to evaluate between group differences in liver histology with p=0.05 as the significance level. We will consider this aim successfully met if in the Hx-001 bioscaffold group shows a significant expression of one or more cytokine and a significant reduction in fibrosis in scored histological sections compared to the Hx-001 alone group.

These results will show that bioscaffolds can improve the performance of Hx-001 in vivo. Phase II studies will focus on defining the mechanism of action with the goal of optimizing activity in non-alcoholic steatohepatitis models and autoimmune hepatitis models. In addition, we will optimize the therapeutic application of the bioscaffold cell combination (e.g. route of administration), and develop production methods for the bioscaffolds and the combination that can support clinical-grade manufacturing. Hx-001 cells are currently in transition to cGMP-type manufacturing. Completion of Phase II will yield a product candidate ready for IND-enabling studies. Hepatx is a venture funded company and will use private investment funds to complete clinical studies and commercialization of Hx-001 bioscaffold products for chronic liver disease.

Use of FGF19 and FGF20 to reduce inflammation and fibrosis in MASH. Cells can be engineered to secrete these proteins. The cells become a kind of slow release system/time release system for these proteins. The cells can be used to alter the PK of the proteins. Cells can be engineered to control secretion of these proteins by controlling transcription or other parts of the synthesis and secretion process. Options for controlling transcription are many and may include: ultrasound controlled promoter, heat shock controlled promoter, optogenetic control of a promoter, ligand induced promoter (e.g., Tet promoter). Optionally, the engineered cells can be combined with a material to facilitate survival in vivo. Implant the cells. Trigger synthesis of FGF protein using ultrasound or light. Monitor disease.

The half-life of many therapeutic proteins is very short, necessitating multiple or large doses. Alternatively, the proteins can be modified to create analogs that address some of the PK issues, but the half-life may still be short and require multiple doses. The area under the curve for many protein therapeutics is too low and correcting that can be expensive, if it can be done.

Gene therapy can achieve a similar result using endogenous cells. Currently, drawbacks to the gene therapy approach include inability to control the genetic modification of the cells and variations in dosage, i.e. how many cells are modified by the therapy. Engineering cells before administration to the patient enables greater control over the genetic modification of the cells, increasing the consistency of the product. The engineered cells cal be accurately quantified enabling more accurate dosing with therapeutic protein.

The main components of this aspect are: nucleic acid encoding the protein or an analog of the protein or any polypeptide that can have a therapeutic effect. More than one protein can be expressed from each cell, i.e. both FGF19 and FGF21; components of the nucleic acid encoding the protein that control its expression, including promoters, polyadenylation signals, enhancers, mRNA cap, polyadenylation, etc.; any cell that can be modified with nucleic acids to express the therapeutic protein of interest; and optionally a carrier to facilitate survival of the modified cells in vivo. Carriers might include collagen, alginate, graphene or other materials. The cells will preferably be easily modified with nucleic acids by lipid-mediated transfection, electroporation, viral transduction, or other means. Preferably these cells will be low immunogenicity or non-immunogenic. Preferably these cells can be successfully cryopreserved.

The nucleic acid can be a plasmid or viral vector engineered with the protein of interest in it or it may be an RNA molecule that can be translated in the cells. Introduction of the nucleic acid can be by any of several means including lipofection, electroporation, or viral infection. The cells can be checked for expression using ELISA assays or other common techniques for measuring protein expression. The cell-nucleic acid combination expressing the protein of interest can then be administered to a patient by any of several routes of administration, including intravenous, subcutaneous, direct injection into tissue, or topical application. The cell-nucleic acid combination can optionally be combined with a material that enhances the ability of the cells to survive in vivo or that may alter the expressed protein in some therapeutically useful way.

Components that can be varied are the protein that is expressed, the ancillary nucleic acids that control the expression of the protein, the cells that are used to express the protein, any materials that are combined with the cell-nucleic acid combination.

Various features and embodiments of the disclosure are illustrated in the following representative examples, which are intended to be illustrative, and not limiting. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1. HX001 Spheres and Development

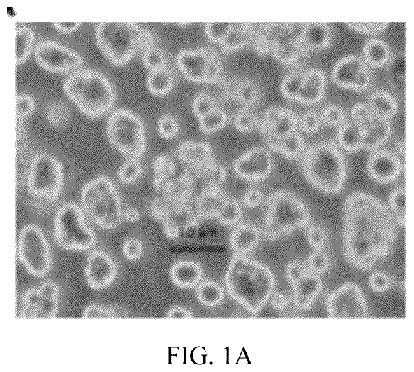

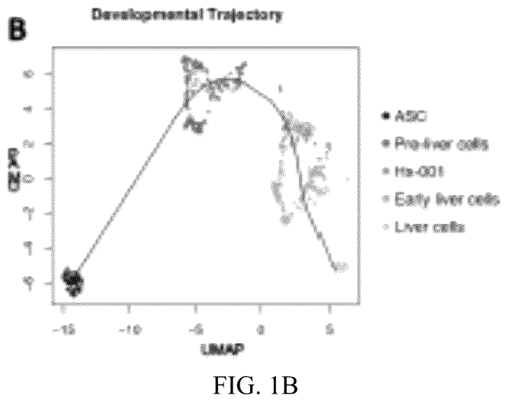

HX001 were prepared using a proprietary suspension sphere method as described by Xu et al. and modified by Hepatx.30 Briefly, adipose-derived stem/stromal cells (ASCs) from lipoaspirate were used to form spheres of cells of approximately 2,000 cells, which were then preconditioned through partial differentiation into the hepatocyte lineage using a two-stage method (FIG. 1A).

Initial exposure to an endoderm medium containing activin A and a WNT agonist was followed by induction of hepatic differentiation by exposure to HGF-containing medium. Importantly, initial formation of ASC spheres has been shown to both accelerate differentiation, as well as yield a more mature phenotype than HLCs derived from induced pluripotent cells.30 Single cell RNA sequencing of our HLCs revealed that the resulting cells resemble transitional hepatoblasts or early hepatocytes rather than mature hepatocytes (FIG. 1B). Expression of genes characteristic of hepatocyte lineage cells, including LDL receptor, GLUL and other genes involved in glycogen synthesis, and certain drug transporters are upregulated, while genes characteristic of the ASC phenotype, including ENG and NT5E are downregulated during differentiation (not shown). Mature hepatocyte genes, such as ALB or ASGR1, are not expressed at this stage. Extensive characterization of these cells in vitro revealed that the cells retain some phenotypic characteristics of the parent ASCs, including the expression of immunomodulatory and trophic genes such as HGF, VEGFA, and indoleamine 2,3-dioxygenase.

### Example 2: Co-Culture with Hx-001 Reduces Inflammatory Cytokine Expression from Macrophages

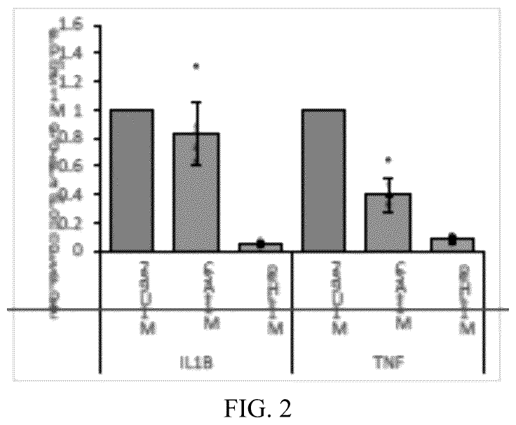

U937 cells were differentiated to macrophages, then treated with interferon gamma and lipopolysaccharide to induce an inflammatory phenotype. Hx-001 or ASCs were added to the cultures using Transwells. After 21 hours of co-culture RNA was analyzed by qPCR. Expression normalized to M1 inflammatory macrophages is shown in FIG. 2.

When co-cultured with activated macrophages, Hx-001 cells can significantly reduce expression of key inflammatory cytokines, including IL-1 beta, IL-6, and TNF alpha (FIG. 2). Differentiation significantly increases this immunomodulatory activity (FIG. 2). Similarly, in co-cultures with HepG2 hepatoma cells that have experienced oxidative damage, Hx-001 cells show the ability to mitigate loss of glutathione and thus oxidative damage.

### Example 3: Hx-001 Cells Reduce Liver Damage, Inflammation, and Fibrosis and Increase Hepatocyte Proliferation in a Murine Model of Liver Injury

C57bl/6 mice were fed ethanol for 10 days, then received a binge of ethanol. Mice then received 2×106 Hx-001 cells IP 6 hours later and 24 hours later. Mice were sacrificed at 48 hours after the ethanol binge. A) ALT and AST measured in serum; B) inflammatory cytokine RNA was analyzed in liver tissue; c) smooth muscle actin and collagen I RNA were measured in liver tissue; d) sections of liver tissue were stained for proliferating cell nuclear antigen (PCNA).

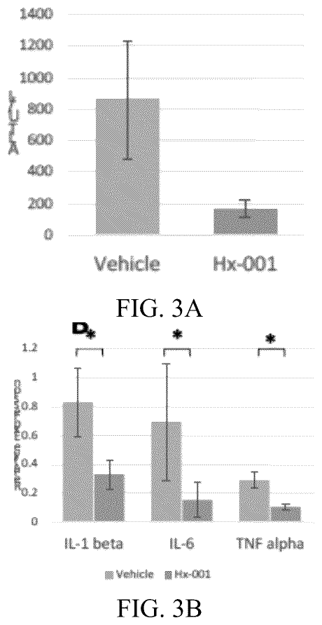

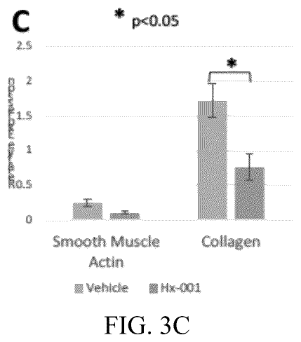

In vivo, results indicate that Hx-001 cells can reduce liver damage, reduce inflammation, and protect hepatocytes from oxidative damage in three different models of liver injury, including a model of alcohol-related hepatitis in mice (FIGS. 3A, 3B and 3C). Mice were subjected to 10 day ethanol feeding plus one ethanol binge on day 11. Galactosamine was administered on days 9-11. Animals were treated with 2×106 Hx-001 cells intraperitoneally 6 hours after the binge and 24 hours after the binge. The animals were sacrificed at 48 hours post-binge. Analysis of blood revealed reduced levels of ALT in cell treated mice (FIG. 3A), reduced inflammatory cytokine expression in liver tissue (FIG. 3B), and reduced expression of smooth muscle actin and collagen I in liver tissue (FIG. 3C).

### Example 4: Biomaterials to Improve Cell Therapy

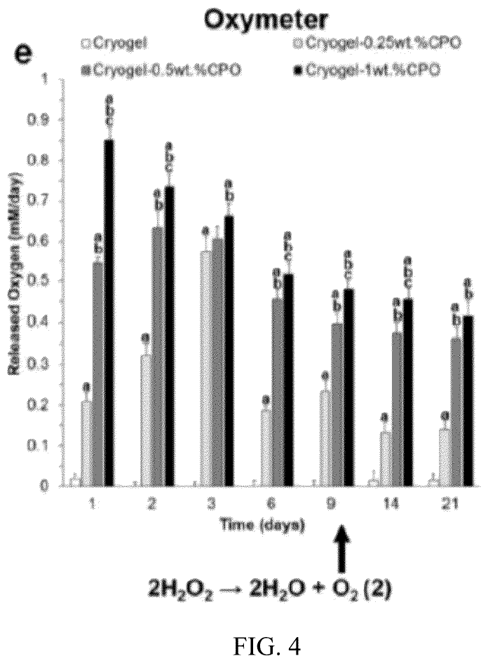

Collagen based cryogel bioscaffolds have the following attributes: (i) macropores that can accommodate cellular aggregates; (ii) co-existing micropores that can facilitate vascular ingrowth; (iii) high surface area-to-volume ratio so a large number of cellular aggregates can be accommodated in a small “footprint”; (iv) 3D matrix that enables an even distribution of cells, thereby preventing clumping and clustering which increase the risk of cell necrosis; and (v) modular rigidity to provide mechanical support for cell, while ensuring sufficient deformability to facilitate implantation at extrahepatic sites; (vi) highly biocompatible with minimal foreign body reaction. The Thakor Group has been able to incorporate additional features into the matrix of the collagen bioscaffold, including CPO. CPO is an oxygen generator that produces oxygen as it gets hydrolyzed. When tested with islets seeded into the cryogel-CPO bioscaffolds, oxygen was shown to be released in a controlled and sustained manner over several days as shown in FIG. 4.

The goal of this proposal is to explore conditions for combining Hx-001 with CPO cryogel bioscaffolds that support and extend the therapeutic function of the cells. The goal will be achieved through iterative cycles of bioscaffold loading with cells and testing, as described in these three Aims: 1) evaluation of optimal oxygen generation and loading of bioscaffolds with HLCs, 2) Evaluation of cell function in combination with bioscaffolds in vitro, and 3) evaluation of the activity of the combination in a mouse model of liver disease. If successful, these preliminary data will form the foundation for the initial commercial development of the Hx-001-bioscaffold combination for the treatment of chronic liver disease.
1. Younossi Z M, Golabi P, Paik J M, Henry A, Dongen C V, Henry L. The global epidemiology of nonalcoholic fatty liver disease (NAFLD) and nonalcoholic steatohepatitis (NASH): a systematic review. Hepatology 2023; 77 (4): 1335-47.
2. Hirode G, Saab S, Wong R J. Trends in the Burden of Chronic Liver Disease Among Hospitalized US Adults. Jama Netw Open 2020; 3 (4): e201997.
3. Bogliotti Y, Roest M V, Mattis A N, et al. Clinical Application of Induced Hepatocyte-like Cells Produced from Mesenchymal Stromal Cells: A Literature Review. Cells 2022; 11 (13): 1998.
4. Yang X, Meng Y, Han Z, Ye F, Wei L, Zong C. Mesenchymal stem cell therapy for liver disease: full of chances and challenges. Cell Biosci 2020; 10 (1): 123.
5. Hammerich L, Tacke F. Hepatic inflammatory responses in liver fibrosis. Nat Rev Gastroenterol Hepatol 2023; 1-14.
6. Wang H J, Gao B, Zakhari S, Nagy L E. Inflammation in Alcoholic Liver Disease. Annu Rev Nutr 2012; 32 (1): 343-68.
7. Kratofil R M, Kubes P, Deniset J F. Monocyte Conversion During Inflammation and Injury. Arteriosclerosis Thrombosis Vasc Biology 2017; 37 (1): 35-42.
8. Chen Y, Yousaf M N, Mehal W Z. Role of sterile inflammation in fatty liver diseases. Liver Res 2018; 2 (1): 21-9.
9. States D of P Toxicology & Therapeutics, University of Kansas Medical Center, Kansas City, Kansas, United, Woolbright B. The impact of sterile inflammation in acute liver injury. J Clin Transl Res 2017; 3 (Suppl 1): 170-88.
10. Zhu M, Hua T, Ouyang T, Qian H, Yu B. Applications of Mesenchymal Stem Cells in Liver Fibrosis: Novel Strategies, Mechanisms, and Clinical Practice. Stem Cells Int 2021; 2021:1-17.
11. Bruno S, Sanchez M B H, Pasquino C, et al. Human Liver-Derived Stem Cells Improve Fibrosis and Inflammation Associated with Nonalcoholic Steatohepatitis. Stem Cells Int 2019; 2019:6351091.
12. Molnar V, Pavelić E, Vrdoljak K, et al. Mesenchymal Stem Cell Mechanisms of Action and Clinical Effects in Osteoarthritis: A Narrative Review. Genes 2022; 13 (6): 949.
13. Sarkar D, Ahamed S M. Mesenchymal stem cells have a crucial role in liver fibrosis treatment: An overview. Sci Archives 2023; 04 (02): 92-8.
14. Hu C, Wu Z, Li L. Mesenchymal stromal cells promote liver regeneration through regulation of immune cells. Int J Biol Sci 2020; 16 (5): 893-903.
15. Park S, Hwang S I, Kim J, et al. The therapeutic potential of induced hepatocyte-like cells generated by direct reprogramming on hepatic fibrosis. Stem Cell Res Ther 2019; 10 (1): 21.
16. Bartneck M, Warzecha K T, Tacke F. Therapeutic targeting of liver inflammation and fibrosis by nanomedicine. Hepatobiliary Surg Nutrition 2014; 3 (6): 364-76.
17. Bogliotti Y, Vander_Roest M, Schuur E. Novel hybrid phenotypes of adipose stromal cell-derived hepatocyte-like cells demonstrate improved immunomodulatory capabilities in vitro and increased effectiveness against liver injury in vivo. Cytotherapy 2022; 24 (5S): S81-2.
18. Winkler S, Hempel M, Brückner S, Tautenhahn H-M, Kaufmann R, Christ B. Identification of Pathways in Liver Repair Potentially Targeted by Secretory Proteins from Human Mesenchymal Stem Cells. Int J Mol Sci 2016; 17 (7): 1099.
19. Hao N-B, Li C-Z, Lü M-H, et al. SDF-1/CXCR4 Axis Promotes MSCs to Repair Liver Injury Partially through Trans-Differentiation and Fusion with Hepatocytes. Stem Cells Int 2015; 2015:960387.
20. Sensebé L, Fleury-Cappellesso S. Biodistribution of Mesenchymal Stem/Stromal Cells in a Preclinical Setting. Stem Cells Int 2013; 2013:678063.
21. Sokal E M, Lombard C A, Roclants V, et al. Biodistribution of Liver-Derived Mesenchymal Stem Cells After Peripheral Injection in a Hemophilia A Patient. Transplantation 2017; 101 (8): 1845-51.
22. Wagner B, Henschler R. Mesenchymal Stem Cells-Basics and Clinical Application II. Adv Biochem Eng Biotechnology 2012; 130:19-37.
23. Lec A S, Inayathullah M, Lijkwan M A, et al. Prolonged survival of transplanted stem cells after ischaemic injury via the slow release of pro-survival peptides from a collagen matrix. Nat Biomed Eng 2018; 2 (2): 104-13.
24. Griffin K H, Fok S W, Leach J K. Strategies to capitalize on cell spheroid therapeutic potential for tissue repair and disease modeling. Npj Regen Medicine 2022; 7 (1): 70.
25. Buravkova L B, Andreeva E R, Gogvadze V, Zhivotovsky B. Mesenchymal stem cells and hypoxia: Where are we? Mitochondrion 2014; 19:105-12.
26. Dhawan A, Chaijitraruch N, Fitzpatrick E, et al. Alginate microencapsulated human hepatocytes for the treatment of acute liver failure in children. J Hepatol 2020; 72 (5): 877-84.
27. Somaiah C, Kumar A, Mawrie D, et al. Collagen Promotes Higher Adhesion, Survival and Proliferation of Mesenchymal Stem Cells. Plos One 2015; 10 (12): c0145068.
28. Nahmias Y, Kramvis Y, Barbe L, et al. A novel formulation of oxygen-carrying matrix enhances liver-specific function of cultured hepatocytes. FASEB J 2006; 20 (14): 2531-3.
29. Razavi M, Primavera R, Kevadiya B D, Wang J, Buchwald P, Thakor A S. A Collagen Based Cryogel Bioscaffold that Generates Oxygen for Islet Transplantation. Adv Funct Mater 2020; 30 (15): 1902463.
30. Xu D, Nishimura T, Zheng M, et al. Enabling Autologous Human Liver Regeneration With Differentiated Adipocyte Stem Cells. Cell Transplantation 2014; 23 (12): 1573-84.
31. Jitraruch S, Dhawan A, Hughes R D, et al. Alginate Microencapsulated Hepatocytes Optimised for Transplantation in Acute Liver Failure. Plos One 2014; 9 (12): e113609.
32. Ho S S, Murphy K C, Binder B Y K, Vissers C B, Leach J K. Increased Survival and Function of Mesenchymal Stem Cell Spheroids Entrapped in Instructive Alginate Hydrogels. Stem Cell Transl Med 2016; 5 (6): 773-81.
33. Mao A S, Özkale B, Shah N J, et al. Programmable microencapsulation for enhanced mesenchymal stem cell persistence and immunomodulation. Proc National Acad Sci 2019; 116 (31): 15392-7.
34. Bochenek M A, Veiseh O, Vegas A J, et al. Alginate encapsulation as long-term immune protection of allogeneic pancreatic islet cells transplanted into the omental bursa of macaques. Nat Biomed Eng 2018; 2 (11): 810-21.
35. Pedraza E, Brady A-C, Fraker C A, et al. Macroporous Three-Dimensional PDMS Scaffolds for Extrahepatic Islet Transplantation. Cell Transplant 2012; 22 (7): 1123-35.
36. Razavi M, Wang J, Thakor A S. Localized drug delivery graphene bioscaffolds for cotransplantation of islets and mesenchymal stem cells. Sci Adv 2021; 7 (47): eabf9221.
37. Primavera R, Razavi M, Kevadiya B D, et al. Enhancing islet transplantation using a biocompatible collagen-PDMS bioscaffold enriched with dexamethasone-microplates. Biofabrication 2021; 13 (3): 035011.
38. Guan N, Liu Z, Zhao Y, Li Q, Wang Y. Engineered biomaterial strategies for controlling growth factors in tissue engineering. Drug Deliv 2020; 27 (1): 1438-51.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the scope of the invention(s) of the disclosure.

What is claimed is:

1. A method comprising administering a composition having a hepatocyte-like cell derived from a mesenchymal stem cell to a patient having NASH or AH.

2. The method of claim 1, wherein the mesenchymal stem cell is an adipose tissue derived mesenchymal stem cell.

3. The method of claim 2, wherein the patient has NASH.

4. The method of claim 2, wherein the patient has AH.

5. The method of claim 2, wherein the hepatocyte-like cell secretes an anti-inflammatory factor.

6. The method of claim 5, wherein the hepatocyte-like cell secretes a pro-regenerative factor.

7. The method of claim 6, wherein the composition further comprises a bioscaffold.

8. The method of claim 7, wherein the bioscaffold has macropores that accommodate the hepatocyte-like cell.

9. The method of claim 8, wherein the bioscaffold facilitates vascular ingrowth.

10. The method of claim 8, wherein the bioscaffold a high surface are to volume ratio.

11. The method of claim 8, wherein the bioscaffold is highly biocompatible.

12. The method of claim 8, wherein the bioscaffold comprises a collagen-based matrix.

13. The method of claim 12, wherein the bioscaffold further comprises a calcium peroxide.

14. The method of claim 13, wherein the calcium peroxide is present in the bioscaffold at up to 1.0% by weight.

15. The method of claim 13, further comprising the step of providing oxygen from the bioscaffold to the hepatocyte-like cell.
